(19) United States
(12) Patent Application Publication
(10) Pub. No.: US 2006/0211907 A1
(43) Pub. Date: Sep. 21, 2006

(54) Extraction of oxygenates from a hydrocarbon stream

(76) Inventors: Johan Pieter de Wet, Vanderbijlpark (ZA); Wilhelmina Jansen, Vanderbijlpark (ZA); Paul Jacobson, Sasolburg (ZA)

Correspondence Address: INDIANAPOLIS OFFICE 27879;BRINKS HOFER GILSON & LIONE, ONE INDIANA SQUARE, SUITE 1600, INDIANAPOLIS, IN, 46204-2033, US

(21) Appl. No.: 10/549,767

(22) PCT Filed: Mar. 10, 2004
(86) PCT No.: PCT/IB04/00656

§ 371 Date: Apr. 3, 2006

Related U.S. Application Data
(60) Provisional application No. 60/453,418, filed on Mar. 10, 2003.
(60) Provisional application No. 60/496,816, filed on Aug. 21, 2003.

(30) Foreign Application Priority Data
Mar. 10, 2003 (ZA) 2003/1937
Aug. 21, 2003 (ZA) 2003/6524

(51) Int. Cl. C07C 7/00

(52) U.S. Cl. 585/864

(57) ABSTRACT

This invention relates to a commercially viable process for extracting oxygenates from a hydrocarbon stream, typically a fraction of the condensation product of a Fischer-Tropsch reaction, while preserving the olefin content of the condensation product. The oxygenate extraction process is a liquid-liquid extraction process that takes place in an extraction column using a mixture of methanol and water as the solvent, wherein an extract from the liquid-liquid extraction is sent to a solvent recovery column from which a tops product comprising methanol, olefins and paraffins is recycled to the extraction column, thereby enhancing the overall recovery of olefins and paraffins.

### BACKGROUND OF THE INVENTION

This invention relates to a process for extracting oxygenates from a hydrocarbon stream.

Many processes for extracting oxygenates from hydrocarbon streams are known. Such extraction methods include hydrogenation, azeotropic distillation, extractive distillation, vapour phase dehydration, liquid phase dehydration and liquid-liquid extraction.

United Kingdom Patent No. 669,313 in the name of California Research Corporation discloses the use of a hydrocarbon condensate from the Fischer-Tropsch process as a feedstock in the production of alkyl benzene. This reference is limited to the use of “high temperature” Fischer-Tropsch processes wherein the Fischer-Tropsch reaction is carried out temperatures of approximately 300° C. and higher, for the production of the hydrocarbon condensate. This reference states that Fischer Tropsch feedstock produced results in poor quality Linear Alkyl Benezene due to odour and wetting problems caused by carbonyl i.e. oxygenate content of the Fischer Tropsch feedstock. Methods suggested for removing the oxygenates include treating the stocks with hot caustic solution or sodium bisulphite followed by extraction with solvents such as methanol, or treatment with boric acid solution to form esters which can be removed by distillation. The preferred method for addressing this problem is by adsorption of oxygenates from the Fischer Tropsch feedstock using activated carbon and silica gel. This process is only feasible for feeds with low oxygenate concentrations. Also, in the example the olefin recovery is less than 25%, i.e. the olefin content is not preserved.

United Kingdom Patent No. 661,916 in the name of Naamlooze Vennootschap De Batafsche Petroleum Maatschappij relates to a method of separating oxygenated compounds from the reaction product of a Fischer-Tropsch reaction by extraction using liquid sulphur dioxide and a paraffinic hydrocarbon flowing in countercurrent to each other. This reference provides that the separation of the oxygenated compounds by extraction with a single solvent such as liquid sulphur dioxide or aqueous methanol has proved difficult and uneconomic in practice.

This invention relates to a commercially viable process for extracting or separating oxygenates from a hydrocarbon stream containing olefins and paraffins, typically the condensation product of a Fischer-Tropsch reaction, while preserving the olefin content of the stream.

### SUMMARY OF THE INVENTION

According to the invention there is provided a commercially viable process for extracting oxygenates from a hydrocarbon stream, typically a fraction of the condensation product of a Fischer-Tropsch reaction, while preserving the olefin content of the condensation product.

The oxygenate extraction process is a liquid-liquid extraction process that preferably takes place in an extraction column using a mixture of methanol and water as the solvent, wherein an extract from the liquid-liquid extraction is sent to a solvent recovery column from which a tops product comprising methanol, olefins and paraffins is recycled to the extraction column, thereby enhancing the overall recovery of olefins and paraffins. A bottoms product from the solvent recovery column may also be recycled to the extraction column.

The solvent preferably has a water content of more than 3% by weight, more preferably a water content of from 5%-15% by weight.

Preferably, a raffinate from the extraction column is sent to a stripper column from which a hydrocarbon feed stream containing more than 90% by weight olefins and paraffins and typically less than 0.2% by weight, preferably less than 0.02% by weight oxygenates exits as a bottoms product. The recovery of olefins and paraffins over the oxygenate extraction process is preferably greater than 70% more preferably greater than 80%, while the olefin/paraffin ratio is at least substantially preserved.

According to another aspect of the invention, the solvent recovery column includes an extract inlet, an upper overhead outlet and a lower bottoms outlet, with a side-draw located above the extract feed point and below the overheads outlet.

The hydrocarbon stream may be the condensation product of a low temperature Fischer-Tropsch reaction carried out at a temperature of 160° C.-280° C., preferably 210° C.-260° C., and a Fischer-Tropsch catalyst, preferably in the presence of a cobalt catalyst to provide a hydrocarbon condensate containing 60 to 80% by weight paraffins and 10 to 30% by weight, typically less than 25% by weight, olefins. The olefins so produced have a high degree of linearity of greater than 92%, typically greater than 95%. The paraffins so produced have a degree of linearity of greater than 92%.

The hydrocarbon condensate product may be fractionated into the C₈ to C₁₆ range, preferably into the C₁₀ to C₁₃ range prior to extraction. Typically, the hydrocarbon stream is a fractionated hydrocarbon condensate product from a low temperature Fischer-Tropsch reaction in the C₁₀ to C₁₃ range containing 10 to 30%, typically less than 25%, by weight olefins with a high degree of linearity of greater than 92%, typically greater than 95%.

### BRIEF DESCRIPTION OF THE DRAWING

FIG. 1 is a graph showing the percent recovery of olefins and paraffins in a solvent recovery column at different solvent to feed ratios, for solvents containing methanol and 0, 3 and 5% water;

FIG. 2 is a graph showing the C10/11 olefin and paraffin recovery in a solvent recovery column at different solvent to feed ratios for solvents containing methanol and 0, 3 and 5% water; and

FIG. 3 is a block diagram of a process of the invention for extracting oxygenates from a hydrocarbon stream.

### DESCRIPTION OF PREFERRED EMBODIMENTS

This invention relates to a process for extracting oxygenates from a fractionated hydrocarbon condensate stream from a Fischer-Tropsch reaction. The hydrocarbon stream with suitably reduced oxygenate content can be used in the production of other chemicals, e.g. a linear alkyl benzene feedstock can be prepared from low temperature Fischer-Tropsch condensate using this invention.

In the Fischer-Tropsch process, synthesis gas (carbon monoxide and hydrogen) obtained either from the gasification of coal or the reforming of natural gas, is reacted over a Fischer Tropsch catalyst to produce a mixture of hydrocarbons ranging from methane to waxes and smaller amounts of oxygenates.

In a low temperature Fischer-Tropsch reaction, the reaction takes place in a slurry bed reactor or fixed bed reactor, preferably a slurry bed reactor, at a temperature in the range of 160° C.-280° C., preferably 210° C.-260° C., and a pressure in the range of 18-50 bar (gauge), preferably between 20-30 bar (gauge), in the presence of a catalyst. The catalyst may include iron, cobalt, nickel or ruthenium. However, a cobalt-based catalyst is preferred for the low temperature reaction. Usually, the cobalt catalyst is supported on an alumina support.

During the low temperature Fischer-Tropsch reaction, a lighter hydrocarbon vapour phase is separated from a liquid phase comprising heavier liquid hydrocarbon products. The heavier liquid hydrocarbon product (waxy products) is the major product of the reaction and may, for example, be hydrocracked to produce diesel and naphtha.

The lighter hydrocarbon vapour phase which comprises gaseous hydrocarbon products, unreacted synthesis gas and water is condensed to provide a “condensation product” which comprises an aqueous phase and a hydrocarbon condensation product phase.

The hydrocarbon condensation product includes ole fins, paraffins in the C₄ to C₂₆ range, and oxygenates including alcohols, esters, aldehydes, ketones and acids.

Typically, a hydrocarbon condensation product for a low temperature Fischer-Tropsch reaction contains 15%-30% by weight olefins, 60%-80% by weight paraffins, and 5%-10% by weight oxygenates. It has, surprisingly, been found that even though this condensation product contains oxygenates and has a low olefin content, it can be used in the production of linear alkyl benzene. However, it is necessary to first extract the oxygenates as these species have a negative effect on the alkylation reaction. There is therefore a need to find a process for extracting oxygenates, but at the same time preserve the olefin content. For the production of linear alkyl benzene, the hydrocarbon condensate product is fractionated into a C₁₀-C₁₃ cut which, by way of example, contains 25% by weight olefins, 68% by weight paraffins and 7% by weight oxygenates. The oxygenate content of this C₁₀-C₁₃ cut can be as high as 15%.

In the prior art, many methods of extracting oxygenates from hydrocarbon streams are suggested. Such removal methods include hydrogenation, azeotropic distillation, extractive distillation, vapour phase dehydration, liquid phase dehydration and liquid-liquid extraction. It has been found that liquid-liquid extraction is a preferred method of oxygenate extraction because, if the correct solvent is selected, the olefin content can be preserved. In liquid-liquid extraction, the solvent can be any polar material that has partial miscibility with the feed stream 14, such as tri-ethanol amine, tri-ethylene glycol with between zero and 20% water, acetonitrile with between 5% and 20% water, acetol, diols, methanol, or ethanol and water. Normally, a high-boiling point solvent is preferred because the solvent recovery steps after extraction require less energy than will be the case for a low-boiling point solvent. However, it has been found that a mixture of methanol and water, which is a low-boiling point solvent, need not suffer from this drawback, because it can be effective at low solvent to feed ratios (this can be lower than 1 if the required oxygenate extraction is not too severe).

Furthermore, one would not expect to be able to use methanol and water as a solvent in a liquid-liquid extraction column to extract oxygenates from the abovementioned hydrocarbon condensate because a study of the different azeotropes with water that exist in the hydrocarbon condensate would lead one to expect that it would not be possible to distil water in a solvent recovery column without azeotroping oxygenates overhead as well. Surprisingly, this turns out not to be the case.

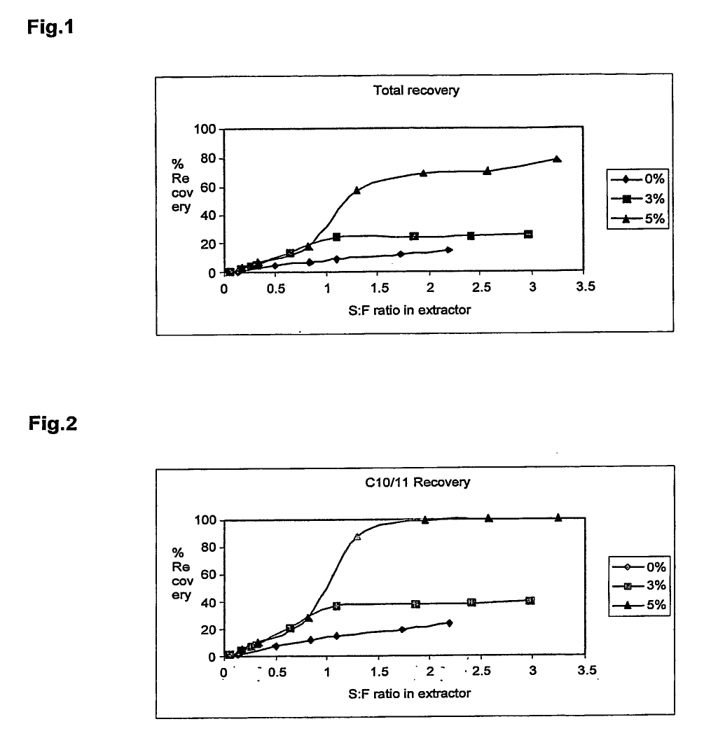

Thus, a further aspect of the invention is that is has been found that a water/methanol solvent, preferably with greater than 3% by weight water content in the liquid-liquid extraction column leads to better recovery of desired products in the solvent recovery column than a dry methanol solvent or a water/methanol solvent with less than 3% by weight water in the liquid-liquid extraction column. This is shown in FIG. 1, from which it can be seen that a methanol/water solvent with 5% by weight water provides as much as 80% recovery of olefins and paraffins in the solvent recovery column. FIG. 2 below shows that almost a 100% recovery of C10/C11 olefins and paraffins in the solvent recovery column is possible.

Thus, according to the invention, typically 90% of the olefins and paraffins are recovered from the liquid-liquid extraction column. The 10% of olefins and paraffins not recovered are sent to the solvent recovery column in the extract from the liquid-liquid extraction column. Up to 60% of the olefins and paraffins in the solvent recovery column are recovered in the overheads product from the solvent recovery column and recycled to the liquid-liquid extraction column. This results in an over-all recovery of olefins and paraffins of more than 90%.

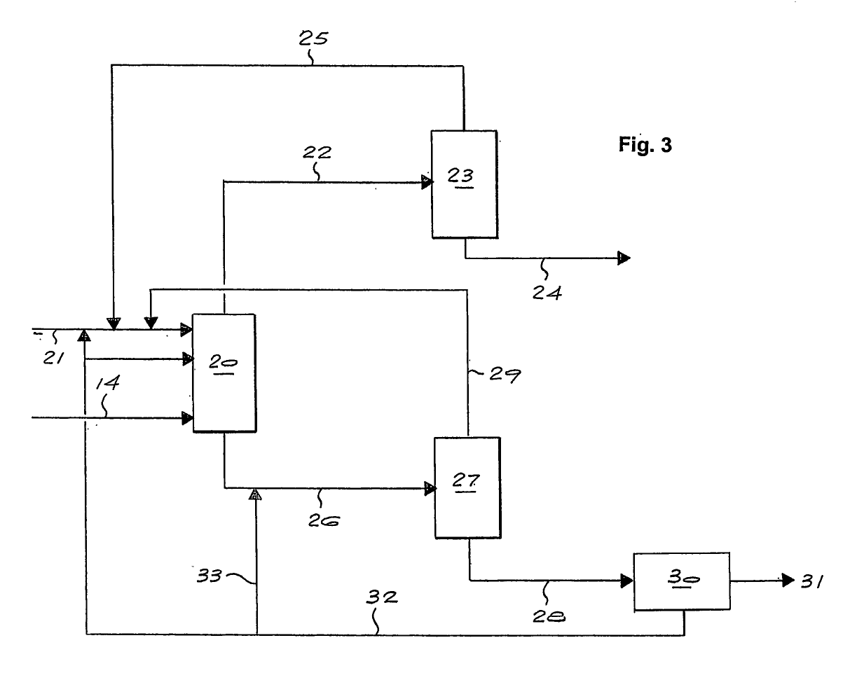

With reference to FIG. 3, a liquid-liquid extraction process of the invention includes an extraction column 20. The fractionated condensation product of a low temperature Fischer-Tropsch reaction described above 14 is fed into the extraction column 20 at, or near, the bottom thereof and a solvent stream 21 comprising a mixture of methanol and water is fed into the extraction column 20 at or near the top thereof. The solvent stream 21 preferably comprises more than 5% by weight, typically 6% by weight, water. The solvent to feed ratio in the solvent stream is low, typically less than 1.5, usually about 1.25.

Raffinate 22 from the top of the extraction column 20, which includes olefins and paraffins and a small amount of solvent, enters a raffinate stripper column 23 and a hydrocarbon product stream comprising more than 90% by weight olefins and paraffins usually up to 99% by weight olefins and paraffins and less than 0.2% by weight, preferably less than 0.02% by weight oxygenates exits as a bottoms product 24. The bottoms product 24, which shows an overall recovery of over 90% of the olefins and paraffins contains more than 20% by weight α-olefins and more than 70% by weight n-paraffins. Thus, the olefin content of the hydrocarbon product (which is intended for use in the production of linear alkyl benzene) has been preserved. A solvent comprising mainly methanol (more than 90% by weight) and low concentrations of water (less than 5% by weight) and olefins/paraffins (less than 5% by weight) exits as a tops product 25 and is returned to the solvent feed stream 21. If it is desired to recover the bottoms product 24 as a vapour stream, this can be done by taking a bottoms vapour stream from the column 20. The liquid product from the column 20 will then be a very small effluent stream.

An extract 26 is drawn from the bottom of the extraction column 20 and is fed to solvent recovery column 27. A tops product 29 from the solvent recovery column 27 comprises over 90% by weight methanol, and olefins and paraffins. Up to 60% of the olefins and paraffins from the extract 26 are recovered to the tops product 29. The tops product is then recycled to the solvent stream 21. The oxygenate content of the tops product 29 can be as low as 50 ppm, depending on the solvent to feed ratio used in the extraction column 20. A bottoms product 28 from the solvent recovery column 27 comprises mainly water, oxygenates and olefins/paraffins. This bottoms product 28 forms two liquid phases that can be decanted in a decanter 30. The organic phase is an oxygenate, olefin and paraffin stream 31, which leaves the process as a product. The aqueous phase is a stream 32, which is recycled to the extraction column 20. This stream 32 can either enter the extraction column at the top along with the solvent stream 21, or slightly lower down the column 20, to prevent the low amount of oxygenates that will be present in this stream from appearing in the raffinate stream 22.

As mentioned above, normally, a high-boiling solvent is preferred for liquid-liquid extraction because the solvent recovery steps after extraction requires less energy than will be the case for a low-boiling solvent. However, it has been found that a mixture of methanol and water, which is a low-boiling solvent, need not suffer from this drawback, because it can be effective at low solvent to feed ratios (this can be lower than 1 if the required oxygenate extraction is not too severe).

A study of the different azeotropes that exist between components in the feed and water would lead one to expect that it would not be possible to distil water overhead in the solvent recovery column 27 without azeotroping oxygenates overhead as well. Surprisingly, this turns out not to be the case. Methanol, which does not form azeotropes with any of the other species present, prevents the water/oxygenate azeotropes from distilling over at the same temperature as the paraffins and olefins. This appears to be due to an extractive distillation effect. As a result, it is possible to distil the paraffins and olefins overhead, while recovering the oxygenates as a bottoms product. This has the effect of enhancing the overall paraffin and olefin recovery of the process, because the overheads 29 of the solvent recovery column 27 is recirculated to the extraction column 20, which means that the paraffins and olefins will be forced to leave the process in the product stream 24.

It is therefore possible to have a hydrocarbon stream 24 with a high overall recovery of olefins and paraffins, without the use of a counter solvent in the extraction column. In this mode of operation, all the methanol, and part of the water (10-50%) are also recovered in the overhead stream 29.

When operating a solvent recovery column in the manner described above, it is to be expected that certain species may become trapped in the column. These species will tend to build up and in the process cause unstable operation of the solvent recovery column. Such species would typically be heavier olefins and paraffins or lighter oxygenates in the present case. Operating the solvent recovery column with a small side draw may prevent the build up of such species and thereby result in much improved operability of the system.

It is also possible to run the extraction column 20 and the solvent recovery column 27 at different methanol/water ratios. This may be desirable because a high water content in the extraction column 20 will lead to increased solvent to feed ratios (because of reduced solubility of oxygenates in the solvent), while a certain amount of water is necessary to achieve the extractive distillation effect in combination with methanol to recover all the paraffins and olefins as overhead products in the solvent recovery column 27. The different methanol/water ratios in the two columns (20 and 27) can be achieved by diverting some of the water in stream 32 to stream 26 by means of a stream 33.

After passing the C₁₀-C₁₃ hydrocarbon feed stream mentioned above through the abovementioned oxygenate extraction process using a mixture of methanol (95% by weight) and water (5% by weight) and a solvent to feed ratio of 1.25, the purified hydrocarbon feed stream contains 22% by weight olefins, 76% by weight paraffins and less than 0.02% by weight oxygenates. Not only does the extraction process extract oxygenates, it also preserves the olefin content of the hydrocarbon feed. The purified hydrocarbon feed stream containing olefins is particularly useful in the production of linear alkyl benzene.

The invention will now be described in more detail with reference to the following non-limiting examples.

### EXAMPLE 1

This example shows a process according to the invention in which oxygenates are removed to a low level from a C₁₀-C₁₃ cut of a hydrocarbon condensate produced by a low temperature Fischer-Tropsch reaction. The extraction column 20 was run at a solvent to feed ratio of 1.25 and a temperature of 50° C. The overall olefin/paraffin recovery in the stream 24 was 89.9%. The olefin/paraffin ratio in the feed was 1:3.7 and 1:3.6 post oxygenate extraction. The olefin/paraffin ratio was therefore substantially preserved. The oxygenate content in stream 24 is only 0.0145%.

Extraction Column 20

| | 14 | | 21 | | 22 | | 26 | |
|---|---|---|---|---|---|---|---|---|
| | Comp | | Comp | | Comp | | Comp | |
| Stream | (wt %) | Flow (kg/hr) | (wt %) | Flow (kg/hr) | (wt %) | Flow (kg/hr) | (wt %) | Flow (kg/hr) |
| Total | 100 | 3000 | 100 | 3750 | 100 | 2530 | 100 | 4220 |
| Total C10-C13 P/O | 92.7 | 2779.7 | 2.16 | 81.0 | 99.1 | 2507.9 | 6.20 | 261.7 |
| Total Oxygenates | 7.3 | 217.7 | 0.000 | 0.000 | 0.0144 | 0.365 | 5.78 | 243.7 |
| Lights and Heavies | 0.057 | 1.7 | 0.004 | 0.144 | 0.0104 | 0.263 | 0.00480 | 0.202 |
| Water | 0.031 | 0.934 | 6.01 | 225.6 | 0.0073 | 0.184 | 5.74 | 242.4 |
| Methanol | 0.000 | 0.000 | 91.7 | 3443.3 | 0.842 | 21.31 | 82.3 | 3472.0 |

Raffinate Stripper Column 23

| | 22 | | 25 | | 24 | |
|---|---|---|---|---|---|---|
| | Comp | Flow | Comp | Flow | Comp | Flow |
| Stream | (wt %) | (kg/hr) | (wt %) | (kg/hr) | (wt %) | (kg/hr) |
| Total | 100 | 2530 | 100 | 30 | 100 | 2500 |
| Total C10-C13 | 99.1 | 2507.9 | 2.63 | 0.793 | 99.97 | 2499.4 |
| P/O | | | | | | |
| Total | 0.0144 | 0.365 | 0.00163 | 0.000491 | 0.0145 | 0.363 |
| Oxygenates | | | | | | |
| Lights and | 0.0104 | 0.263 | 0.0887 | 0.0267 | 0.00808 | 0.202 |
| Heavies | | | | | | |
| Water | 0.0073 | 0.184 | 1.52 | 0.456 | 0.00115 | 0.0288 |
| Methanol | 0.842 | 21.31 | 95.4 | 28.7 | 0.000 | 0.000 |

Solvent Recovery Column 27

| | 26 | | 29 | | 28 | |
|---|---|---|---|---|---|---|
| Stream | Comp (wt %) | Flow (kg/hr) | Comp (wt %) | Flow (kg/hr) | Comp (wt %) | Flow (kg/hr) |
| Total | 100 | 4220 | 100 | 3584 | 100 | 636 |
| Total C10-C13 P/O | 6.20 | 261.7 | 2.37 | 85.1 | 27.6 | 175.8 |
| Total Oxygenates | 5.78 | 243.7 | 0.00140 | 0.0503 | 42.0 | 267.0 |
| Lights and Heavies | 0.00480 | 0.202 | 0.00747 | 0.268 | 0.00279 | 0.0177 |
| Water | 5.74 | 242.4 | 1.30 | 46.8 | 29.3 | 186.6 |
| Methanol | 82.3 | 3472.0 | 96.2 | 3451.9 | 1.04 | 6.63 |

### EXAMPLE 2

This example shows a process according to the invention in which oxygenates are removed to a low level from a C₁₀-C₁₃ cut of a hydrocarbon condensate produced by a low temperature Fischer-Tropsch reaction, with a higher feed concentration of oxygenates (12,36% by weight) than in Example 1. The extraction column 20 was run at a solvent to feed ratio of 2:1 and a temperature of 50° C. The overall olefin/paraffin recovery in the stream 24 was 91.4%. Once again, the olefin/paraffin ratio was substantially preserved. The oxygenate content in stream 24 is only 0.0154%.

Extraction Column 20

| | 14 | | 32 | | 21 | | 22 | | 26 | |
|---|---|---|---|---|---|---|---|---|---|---|
| | Comp | Flow | Comp | Flow | Comp | Flow | Comp | Flow | Comp | Flow |
| Stream | (wt %) | (kg/hr) | (wt %) | (kg/hr) | (wt %) | (kg/hr) | (wt %) | (kg/hr) | (wt %) | (kg/hr) |
| Total | 100 | 3000 | 0 | 0 | 100 | 6000 | 100 | 2430 | 100 | 6570 |
| Total C10-C13 P/O | 87.6 | 2627.2 | 0 | 0 | 5.91 | 354.9 | 98.9 | 2403.4 | 8.81 | 578.7 |
| Total Oxygenates | 12.36 | 370.7 | 0 | 0 | 0.008 | 0.485 | 0.01529 | 0.4 | 5.64 | 370.7 |
| Lights and Heavies | 0.0698 | 2.0930 | 0 | 0 | 0.000 | 0.002 | 0.07472 | 1.8159 | 0.00425 | 0.279 |
| Water | 0.0000 | 0.000 | 0 | 0 | 11.75 | 705.3 | 0.00314 | 0.0762 | 10.73 | 705.2 |
| Methanol | 0.000 | 0.000 | 0 | 0 | 82.3 | 4939.4 | 1.02 | 24.8 | 74.8 | 4914.6 |

Raffinate Stripper Column 23

| | 22 | | 25 | | 24 | |
|---|---|---|---|---|---|---|
| | | Flow | | Flow | | Flow |
| Stream | Comp (wt %) | (kg/hr) | Comp (wt %) | (kg/hr) | Comp (wt %) | (kg/hr) |
| Total | 100 | 2430 | 100 | 26 | 100 | 2405 |
| Total C10-C13 P/O | 98.9 | 2403.4 | 3.94 | 1.017 | 99.92 | 2402.408 |
| Total Oxygenates | 0.01529 | 0.372 | 0.00113 | 0.00029 | 0.0154 | 0.37139 |
| Lights and Heavies | 0.075 | 1.8159 | 0.0000 | 0.0000 | 0.076 | 1.8159 |
| Water | 0.00314 | 0.0762 | 0.294 | 0.0761 | 0.000006 | 0.0002 |
| Methanol | 1.02 | 24.8 | 95.8 | 24.7 | 0.001 | 0.0 |

Solvent Recovery Column 27

| | 26 | | 25 | | 29 | | 28 | |
|---|---|---|---|---|---|---|---|---|
| | | Flow | | Flow | | Flow | | Flow |
| Stream | Comp (wt %) | (kg/hr) | Comp (wt %) | (kg/hr) | Comp (wt %) | (kg/hr) | Comp (wt %) | (kg/hr) |
| Total | 100 | 6570 | 100 | 26 | 100 | 5400 | 100 | 1195 |
| Total C10-C13 P/O | 8.81 | 578.7 | 3.94 | 1.017 | 6.57 | 354.9 | 18.8 | 224.8 |
| Total Oxygenates | 5.64 | 370.7 | 0.00060 | 0.00015 | 0.00000 | 0.0 | 31.0 | 370.7 |
| Lights and Heavies | 0.00425 | 0.279 | 0.0000 | 0.0000 | 0.00005 | 0.002 | 0.02317 | 0.2770 |
| Water | 10.73 | 705.2 | 0.294 | 0.0761 | 1.96 | 105.8 | 50.1 | 599.5 |
| Methanol | 74.8 | 4914.6 | 95.8 | 24.7 | 91.5 | 4939.3 | 0.00 | 0.05 |

1-16. (canceled)

17. A process for extracting oxygenates from a hydrocarbon stream containing a range of hydrocarbons in the C₈ to C₁₆ range, the process including the step of extracting the oxygenates in a liquid-liquid extraction process using a mixture of methanol and water as the solvent,
wherein an extract from the liquid-liquid extraction is sent to a solvent recovery column from which a tops product comprising methanol, olefins and paraffins is recycled to the extraction step, thereby enhancing the overall recovery of olefins and paraffins.

18. The process according to claim 17, wherein the aqueous phase of a bottoms product from the solvent recovery column is recycled to the extraction step.

19. The process according to claim 17, wherein the extraction step takes place in an extraction column.

20. The process according to claim 17, wherein the solvent introduced to the extraction step has a water content of more than 3% by weight.

21. The process according to claim 20, wherein the solvent has a water content of from 5%-15% by weight.

22. The process according to claim 17, wherein the olefin/paraffin ratio of the hydrocarbon stream is substantially preserved after the extraction step.

23. The process according to claim 19, wherein a raffinate from the extraction column is sent to a stripper column from which a hydrocarbon feed stream containing more than 90% by weight olefins and paraffins and less than 0.2% by weight oxygenates exits as a bottoms product.

24. The process according to claim 23, wherein the bottoms product contains less than 0.02% by weight oxygenates.

25. The process according to claim 17, wherein the recovery of olefins and paraffins over the oxygenate extraction step is greater than 70%.

26. The process according to claim 25, wherein the recovery of olefins and paraffins over the oxygenate extraction step is greater than 80%.

27. The process according to claim 17, wherein the solvent recovery column includes an extract inlet, an upper overhead outlet and a lower bottoms outlet, with a side-draw located above the extract feed point and below the overheads outlet.

28. The process according to claim 17, wherein the hydrocarbon stream is the fractionated condensate product from a low temperature Fischer-Tropsch reaction.

29. The process according to claim 17, wherein the hydrocarbon stream contains 5-15% by weight oxygenates.

30. The process according to claim 17, wherein the fractionated hydrocarbon condensate product is in the C₁₀ to C₁₃ range.
